(19) United States
(12) Patent Application Publication
(10) Pub. No.: US 2019/0000664 A1
(43) Pub. Date: Jan. 3, 2019

(54) DRY EYE COMPRESS WITH HEAT REFLECTIVE COVER

(71) Applicant: Solana Health, Inc., Del Mar, CA (US)

(72) Inventor: David McMahon, Del Mar, CA (US)

(73) Assignee: Solana Health, Inc., Del Mar, CA (US)

(21) Appl. No.: 15/639,929

(22) Filed: Jun. 30, 2017

(51) Int. Cl. A61F 7/02

(52) U.S. Cl. CPC A61F 7/02; A61F 2007/0004

(57) ABSTRACT

The disclosure concerns an improved system for administering moisture therapy to a patient, the system including: a conventional moisture therapy compress, and a therapeutically adaptive cover configured to contain the conventional moisture therapy compress and further adapted to communicate a therapeutic delivery of hot or cold moisture through a fluid permeable material to a treatment site of a patient. The therapeutically adaptive cover communicates hot or cold moisture therapy while isolating the compress from the treatment site, allowing for re-use of the compress by way of replacing the cover, or washing the cover prior to subsequent use for maintaining optimum sterility and cleanliness. Other features and advantages are disclosed in the appended detailed description of the preferred embodiments.

### BACKGROUND

### Field of the Invention

This invention relates to dry eye compresses; and more particularly, to a dry eye compress with a heat reflective cover.

### Description of the Related Art

U.S. Pat. No. 9,445,939 to Bruder et al. (“the '939 patent”) describes a conventional device and method for providing moisture therapy to a subject by applying a moist therapy compress against a treated body portion. The moist therapy compress includes a fluid-permeable shell, a flexible backing fastened to the shell to define an enclosure, and a hydrophilic zeolite fill granules loosely contained within the enclosure. The therapy compress is exposed to a source of moisture to cause absorption of water into the hydrophilic zeolite, and the moisture is delivered from the hydrophilic zeolite through the fluid permeable shell to the treated body portion. The entire contents of the '939 patent are hereby incorporated by reference.

Problems associated with such conventional hot/cold moisture therapy compresses include: limited moisture at the treated tissue; limited treatment time (heat escapes quickly); risk of damage to skin cells at the treatment site; incapable of machine wash; exposure to elements drives excessive wear/damage to the compress unit; and allergic reactions at the treatment site; among others.

There is a continued need for an improved moisture therapy compress configured to address these and other limitations in the art.

### SUMMARY

The disclosure concerns an improved system for administering moisture therapy to a patient, the system including: a conventional moisture therapy compress, and a therapeutically adaptive cover configured to contain the conventional moisture therapy compress and further adapted to communicate a therapeutic delivery of hot or cold moisture through a fluid permeable material to a treatment site of a patient. The therapeutically adaptive cover communicates hot or cold moisture therapy while isolating the compress from the treatment site, allowing for re-use of the compress by way of replacing the cover, or washing the cover prior to subsequent use for maintaining optimum sterility and cleanliness. Other features and advantages are disclosed in the appended detailed description of the preferred embodiments.

### BRIEF DESCRIPTION OF THE DRAWINGS

FIG. 1 shows a system for moisture therapy in accordance with an illustrated embodiment.

FIG. 2 shows the system according to FIG. 1, with the conventional moisture therapy compress being partially inserted and partially removed.

FIG. 3 shows a conventional moisture therapy compress.

FIG. 4 shows a front side of the cover in accordance with the illustrated embodiment.

FIG. 5 shows a rear side of the cover in accordance with the illustrated embodiment.

FIG. 6 shows a plot illustrating percent relative humidity as a function of time with respect to each of a conventional moisture therapy compress and a system in accordance with the illustrated embodiment.

### DETAILED DESCRIPTION

In the following description, for purposes of explanation and not limitation, details and descriptions are set forth in order to provide a thorough understanding of the embodiments of the invention. However, it will be apparent to those skilled in the art that the present invention may be practiced in other embodiments, including certain variations or alternative combinations that depart from these details and descriptions yet arrive at substantially similar results.

In the art of moisture therapy, including that related to compresses for the treatment of dry eye syndrome, it is known that hot and cold moisture therapy can be useful for to treating dry eye. However, clinical use of such moisture therapy compresses is significantly limited due to several problems: (i) conventional moisture therapy compresses are prone to contamination resulting from contact with a first patient, such that a second patient may receive a trans-communicable disease or pathogen infection; (ii) conventional moisture therapy compresses are not machine washable due to the zeolite fill or other chemically active contents within the compress; and (iii) it is not cost effective to provide such conventional compresses for single-use and disposal. Accordingly, conventional moisture therapy compresses have not been well integrated into clinical practice. In addition, individual consumers may require a means to wash or sterilize a moisture therapy compress to prevent spread of pathogens to other family members, or excessive buildup and festering of contaminants to one's personal moisture therapy device.

Accordingly, the instant disclosure presents a solution, including a machine-washable cover configured to nest with an inserted device. Collectively, the cover and inserted device form a system for moisture therapy. It has been discovered that the cover can be optimized through design and material selection to provide moisture for a longer duration than conventional moisture therapy compresses.

The system for moisture therapy can be used to treat dry eye syndrome (DES), meibomian gland dysfunction (MGD), temporomandibular joint disorders (TMJ), sinus pressure, tension, stress, conjunctivitis, sty, and other similar discomforts.

The cover is manufactured from an antimicrobial material or a material treated with an antimicrobial agent for preventing the proliferation of bacteria and fungi. Examples of antimicrobial materials for manufacturing the cover include but are not limited to: Cottons, Polyesters, and other textiles and non-woven fabrics treated with antimicrobial chemicals, including but not limited to: Silver zeolite, titan oxide, silver silicate, soluble glass powder with metallic ions, silver sulphonate, iron-phtalozyanat, copper sulphonate, Biozol, Thimol, Alkylenbisphenol sodium salt, or Chitosan. In the preferred embodiment, the antimicrobial material chosen is a composition containing ultra-fine silver crystals, such as but not limited to, Silpure silver antimicrobial treatment (http://www.ultra-fresh.com/silpure/).

At least a rear surface, termed herein a “first surface”, which is adapted to contact the skin of a patient, is fabricated from such an antimicrobial material or is otherwise treated with an antimicrobial agent as described above.

The conventional moisture therapy compress is thoroughly described in U.S. Pat. No. 9,445,939 to Bruder et al. (“the '939 patent”); the entire contents of which are hereby incorporated by reference. In sum, a conventional moisture therapy compress in accordance with the '939 patent includes: a flexible first shell, the first shell forming at least one enclosure, and being microwave compatible for repeated microwave heating; a plurality of hydrophilic zeolite fill granules loosely contained within the at least one enclosure for delivery of moist heat therapy from the hydrophilic zeolite fill granules through the flexible outer shell, the hydrophilic zeolite fill granules retaining at least one antimicrobial metal material disposed therein; wherein the hydrophilic zeolite fill granules are microwave compatible and substantially retain said at least one antimicrobial metal material therein upon multiple exposures to microwave irradiation, but allow repeated absorption and dissipation of moisture to and from the hydrophilic zeolite fill granules.

However, this disclosure adds to the disclosure of the '939 patent a machine-washable therapeutically active cover for use with the conventional moisture therapy compress, wherein the therapeutically adaptive cover communicates hot or cold moisture therapy while isolating the conventional compress from the treatment site, allowing for re-use of the compress by way of replacing the cover, or washing the cover prior to subsequent use for maintaining optimum sterility and cleanliness.

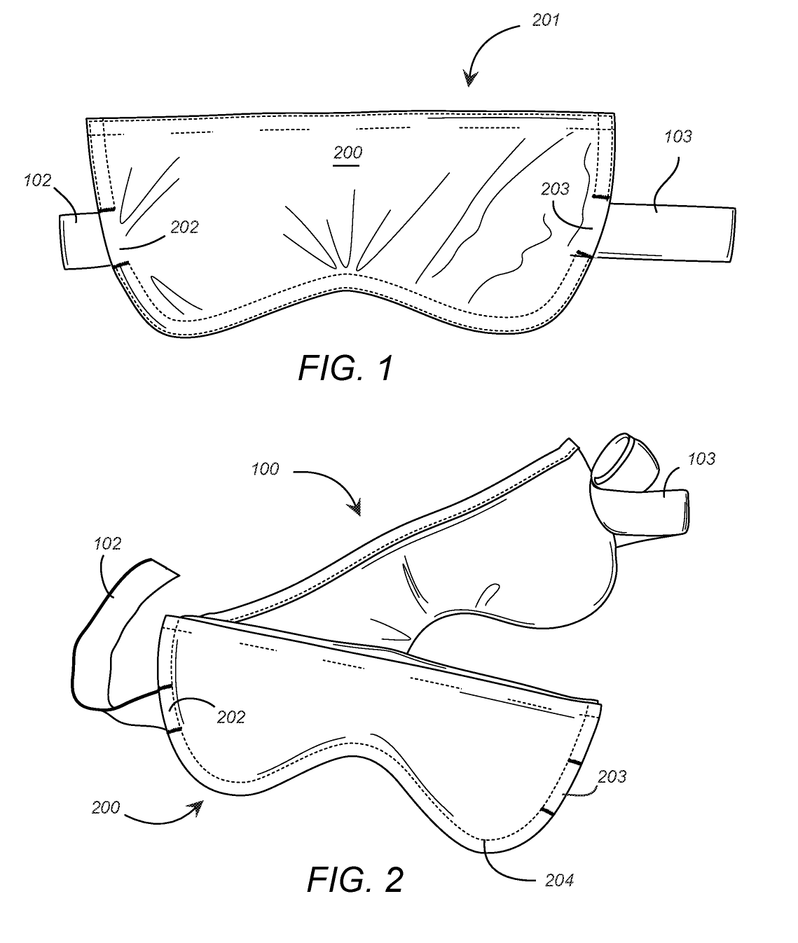

Now turning to the drawings, FIG. 1 shows a system for moisture therapy, the system showing a therapeutically active cover 200 having a first slot 202 disposed at a first side of the cover, and a second slot 203 disposed at a second side of the cover opposite the first side. The first and second slots 202; 203 are each adapted to receive one of a first strap 102, and a second strap 103 associated with a conventional moisture therapy compress. In this regard, the straps of the compress extend through the slots of the cover. In addition, the cover includes a first opening 201 extending along an edge of the cover, wherein the first opening is adapted to receive a body of the conventional compress therethrough. The primary slot may be further adapted with a closing mechanisms (not shown), such as a zipper, buttons, Velcro and the like.

FIG. 2 shows the system according to FIG. 1, with the conventional moisture therapy compress 100 being partially inserted and partially removed. The second strap 103 of the moisture therapy compress 100 is shown at a right side with respect to the illustration. The second strap 103 is configured to insert and extend through the second slot 203 of the cover 200. For purposes herein, the term “cover” is synonymous with “second shell”, wherein the second shell (or cover) comprises a first opening 201 extending along an edge of the second shell. As described above, the first opening is configured to receive the body of the conventional moisture therapy compress. The second shell is generally fabricated from a flexible material, and is thereby a flexible second shell, meaning that it may be deformed or bent without breaking.

The cover 200 may be formed from two sheets, each of which forming one of a front and a back surface of the cover, wherein the two sheets are sewn together along a portion of the periphery thereof to form a seam 204 or capping (not shown). In this regard, the sheets may comprise the same or distinct materials. The rear facing sheet (the sheet configured to contact tissue of a patient) may be formed from an antimicrobial material, or otherwise treated with an antimicrobial agent. Alternatively, the entire cover may be fabricated from an antimicrobial material or otherwise treated with an antimicrobial agent.

The material chosen to manufacture the cover should be one that is permeable to moisture, such that hot or cold moisture may be communicated to the patient. Examples of such materials may include but are not limited to: textiles and nonwoven fabrics.

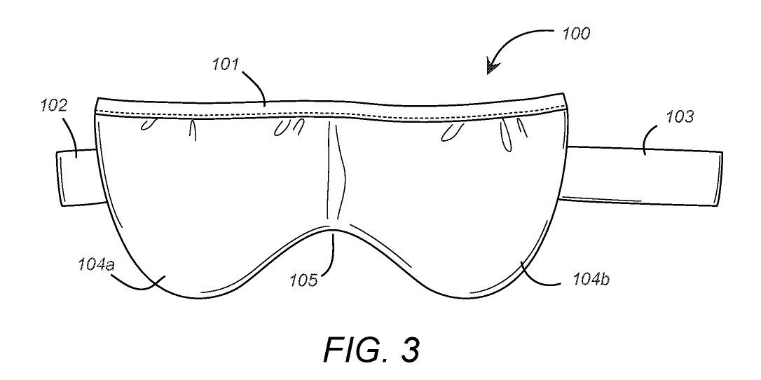

FIG. 3 shows a conventional moisture therapy compress 100. The moisture therapy compress generally includes a first eye portion 104a, a second eye portion 104b adjacent to the first eye portion, the first and second eye portions collectively forming a body 104 of the compress 100 and are configured to contact the tissue of a patient. The compress further includes a first strap 102 disposed at a first side of the body 104, and a second strap 103 disposed at a second side of the body 104 opposite the first side. A notch 105 is provided at a bottom edge of the compress between the first and second eye portions; the notch is configured to contact and conform about a patient's nose. An upper edge of the compress body includes a body capping 101 configured to permanently seal a body aperture used to fill the zeolite granules within a volume contained by the compress body.

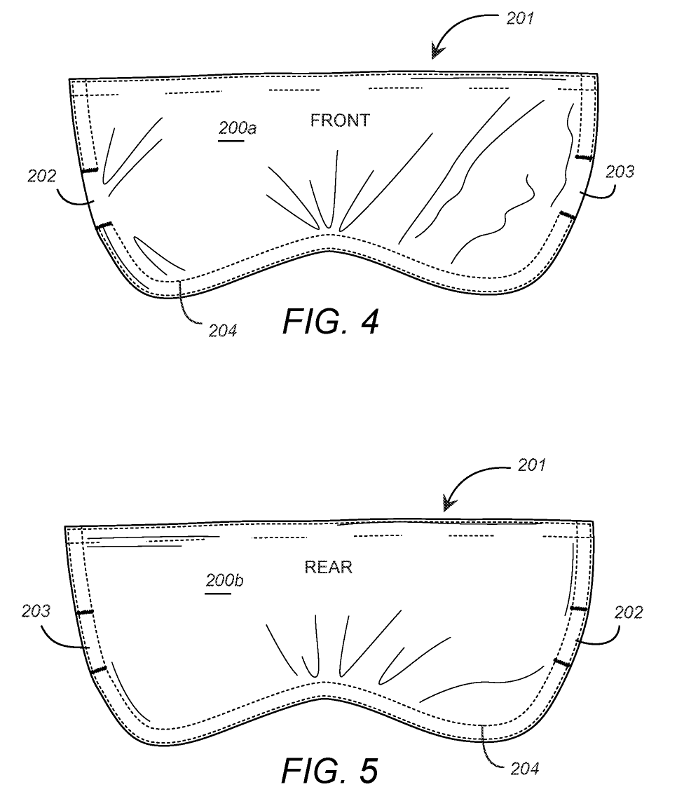

FIGS. 4-5 show a front and rear side of the cover, respectively. The cover includes a first sheet 200a configured to form a front side of the cover, and a second sheet 200b configured to form a rear side of the cover. The cover includes a first opening 201 extending along an upper edge of the cover, the first opening is configured to receive a compress therein. The cover further includes a first slot 202 and a second slot 203, each of the first and second slots being individually configured to receive one of the first and second straps of the compress extending therethrough. A stitching 204 extends about a portion of the cover except at the first and second slots and the first opening, the stitching configured to attach the first and second sheets to form the cover.

The rear side of the cover may be fabricated from a moisture wicking fabric, such as polyester materials.

While two sheets have been disclosed for forming the cover of the moisture therapy system in this illustrated embodiment, it is contemplated that three or more sheets may be similarly implemented provided moisture is capable of being communicated therethrough among other requirements as described or inherent within the instant disclosure.

Now, while the illustrated embodiment has been disclosed in order to enable those with skill in art to make the moisture therapy system including a moisture therapy compress and a therapeutically active cover, it should be recognized by the same that other features and variations may be similar implemented without undue experimentation in order to achieve the same or substantially similar results. Accordingly, the instant disclosure is intended to be enabling only, and not limiting in any way with regard to the spirit and scope of the invention.

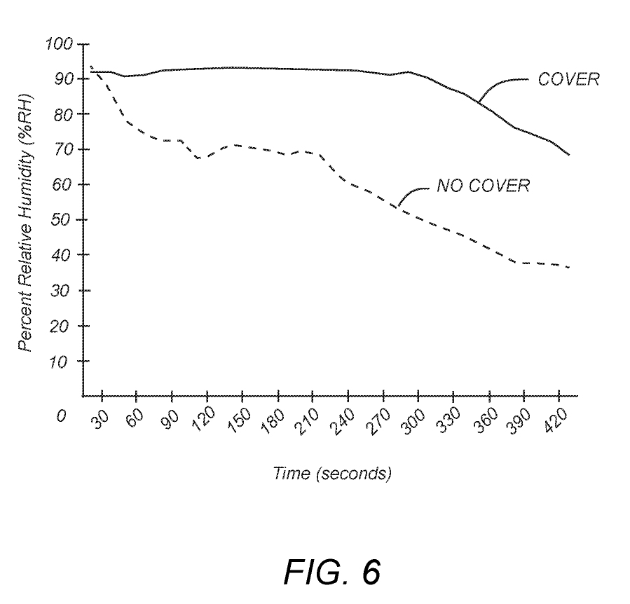

Further, significant experimentation was performed and data obtained as illustrated in FIG. 6. The data shows the relation of percent relative humidity with respect to time in seconds. Data was gathered using a conventional moisture therapy compress only (labeled: NO COVER) and repeated under the same conditions except with the addition of the cover as described herein (labeled: COVER). The results show that the system including a moisture therapy compress and a cover provides moisture, in terms of percent relative humidity, for a longer duration. Accordingly, one benefit of the described system with cover includes extended delivery of moisture therapy.

### REFERENCE SIGNS LIST

100 compress
101 capping
102 first strap
103 second strap
104 compress body
104a first eye portion
104b second eye portion
105 notch
200 cover/second shell
200a first sheet
200b second sheet
201 first opening
202 first slot
203 second slot
204 stitching

I claim:

1. A moisture therapy compress, comprising:
a flexible first shell, the first shell forming at least one enclosure, and being microwave compatible for repeated microwave heating;
a plurality of hydrophilic zeolite fill granules loosely contained within the at least one enclosure for delivery of moist heat therapy from the hydrophilic zeolite fill granules through the flexible outer shell, the hydrophilic zeolite fill granules retaining at least one antimicrobial metal material disposed therein;
wherein the hydrophilic zeolite fill granules are microwave compatible and substantially retain said at least one antimicrobial metal material therein upon multiple exposures to microwave irradiation, but allow repeated absorption and dissipation of moisture to and from the hydrophilic zeolite fill granules;
wherein the moisture therapy compress further comprises:
a flexible second shell, the second shell comprising a first opening extending along an edge thereof, the flexible second shell forming a cover for enclosing the flexible first shell therein.

2. The moisture therapy compress of claim 1, the flexible second shell comprising a pair of slots, the slots each being configured to receive one of a pair of straps connected to the flexible first shell.

3. The moisture therapy compress of claim 1, the flexible second shell comprising a moisture wicking fabric at a rear surface thereof, wherein the rear surface is configured to contact skin of a patient.
